(19) United States
(12) Patent Application Publication
(10) Pub. No.: US 2010/0099690 A1
(43) Pub. Date: Apr. 22, 2010

(54) XANTHINE DERIVATIVES AS SELECTIVE HM74A AGONISTS

(76) Inventors: Jag Paul Heer, Essex (GB); Ian Edward David Smith, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/063,433

(22) PCT Filed: Aug. 8, 2006
(86) PCT No.: PCT/EP06/07876

§ 371 Date: Feb. 9, 2008

(30) Foreign Application Priority Data
Aug. 10, 2005 (GB) 0516462.9

(51) Int. Cl. A61K 31/522; C07D 473/04; A61P 3/00; A61P 9/00

(52) U.S. Cl. 514/263.34; 544/267; 514/247

(57) ABSTRACT

The present invention relates to compounds which are xanthine derivatives, processes for the manufacture of said derivatives, pharmaceutical formulations containing the active compounds and the use of the compounds in therapy, for example, in the treatment of diseases where under-activation of the HM74A receptor contributes to the disease or where activation of the receptor will be beneficial.

The present invention relates to compounds which are xanthine derivatives, processes for the manufacture of said derivatives, pharmaceutical formulations containing these compounds and the use of the compounds in therapy, for example, in the treatment of diseases where under-activation of the HM74A receptor contributes to the disease or where activation of the receptor will be beneficial.

Dyslipidaemia is a general term used to describe individuals with aberrant lipoprotein profiles. Clinically, the main classes of compounds used for the treatment of patients with dyslipidaemia, and therefore at risk of cardiovascular disease are the statins, fibrates, bile-acid binding resins and nicotinic acid. Nicotinic acid (Niacin, a B vitamin) has been used clinically for over 40 years in patients with various forms of dyslipidaemia. The primary mode of action of nicotinic acid is via inhibition of hormone-sensitive triglyceride lipase (HSL), which results in a lowering of plasma non-esterified fatty acids (NEFA) which in turn alters hepatic fat metabolism to reduce the output of LDL and VLDL (low and very low density lipoprotein). Reduced VLDL levels are thought to lower cholesterol ester transfer protein (CETP) activity to result in increased HDL (high density lipoprotein) levels which may be the cause of the observed cardiovascular benefits. Thus, nicotinic acid produces a very desirable alteration in lipoprotein profiles; reducing levels of VLDL and LDL whilst increasing HDL. Nicotinic acid has also been demonstrated to have disease modifying benefits, reducing the progression and increasing the regression of atherosclerotic lesions and reducing the number of cardiovascular events in several trials.

The observed inhibition of HSL by nicotinic acid treatment is mediated by a decrease in cellular cyclic adenosine monophosphate (cAMP) caused by the G-protein-mediated inhibition of adenylyl cyclase. Recently, the G-protein coupled receptors HM74 and HM74A have been identified as receptors for nicotinic acid (PCT patent application WO02/84298; Wise et. al. J Biol. Chem., 2003, 278 (11), 9869-9874). The DNA sequence of human HM74A may be found in Genbank; accession number AY148884. Two further papers support this discovery, (Tunaru et. al. Nature Medicine, 2003, 9(3), 352-255 and Soga et. al. Biochem Biophys Res Commun., 2003, 303 (1) 364-369), however the nomenclature differs slightly. In the Tunaru paper what they term human HM74 we term HM74A and in the Soga paper HM74b is identical to HM74A. Cells transfected to express HM74A and/or HM74 gain the ability to elicit Gᵢ G-protein mediated responses following exposure to nicotinic acid. In mice lacking the homologue of HM74A (m-PUMA-G) nicotinic acid fails to reduce plasma NEFA levels.

Certain xanthine derivatives have been synthesised and disclosed in the prior art. For example, EP0389282 discloses xanthine derivatives as potential mediators of cerebrovascular disorders. A range of xanthine derivatives were identified as adenosine receptor antagonists by Jacobson et. al. in J. Med. Chem., 1993, 36, 2639-2644.

We now present a group of xanthine derivatives which are selective agonists of the nicotinic acid receptor HM74A and are thus of potential benefit in the treatment, prophylaxis and suppression of diseases where under-activation of this receptor either contributes to the disease or where activation of the receptor will be beneficial.

### SUMMARY OF THE INVENTION

The present invention provides xanthine derivatives and the use of these derivatives in therapy, for example, in the treatment of diseases where under-activation of the HM74A receptor contributes to the disease or where activation of the receptor will be beneficial. For example, in treatment of diseases of lipid metabolism including dyslipidaemia or hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia. As such, the compounds may also find favour as therapeutics for coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity. The compounds may also be of use in the treatment of inflammatory diseases or conditions, as set out further below.

Intermediates, formulations, methods and processes described herein form further embodiments of the invention.

### DETAILED DESCRIPTION OF THE INVENTION

According to one aspect of this invention we provide at least one chemical entity selected from compounds of formula (I)

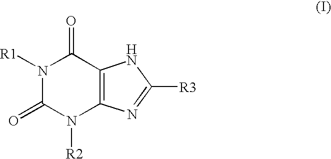

and pharmaceutically acceptable derivatives thereof, wherein
R¹ represents a group selected from
(CH₂)_{q}-cycloalkenyl, —(CH₂)_{q}-aryl and —(CH₂)_{q}-heteroaryl;
Wherein if q is an integer selected from 1 or 2, the R¹ ring may be substituted by one or more groups independently selected from
C₁₋₆alkyl, C₂₋₄alkenyl, C₂₋₆alkynyl, halogen, —NH₂, —(CH₂)_{q}—(O)ₚ—(CH₂)_{q}—N(R⁵)C(O)OR⁸, —(CH₂)_{q}—N(R⁵)C(O)R⁸, —(CH₂)_{q}—(O)ₚ—(CH₂)_{q}—C(O)NR⁵R⁶, —(CH₂)_{q}—N(R⁵)C(O)N(R⁵)R⁶, —(CH₂)_{q}—C(O)N((CH₂)ₘOH)R⁵, —(CH₂)_{q}—N(R⁵)—S(O)₂R⁸, —CH₂—S(O)₂N(R⁵)R⁶, —OCF₃, —OCH(F)₂, —OCH₂F, —R⁸CN, CN and —SO₂R⁹;
Wherein if q is 0, the R¹ ring may be substituted by one or more groups independently selected from C₁₋₆alkyl, C₂₋₆alkenyl, C₂₋₆alkynyl, halogen, —NH₂, —(CH₂)_{q}(O)ₚ—(CH₂)_{q}N(R⁵)C(O)OR⁸, —(CH₂)_{q}—N(R⁵)C(O)R⁸, —(CH₂)_{q}—(O)ₚ—(CH₂)_{q}—C(O)NR⁵R⁶, —(CH₂)_{q}—N(R⁵)C(O)N(R⁵)R⁶, —(CH₂)_{q}—C(O)N((CH₂)ₘOH)R⁵, —(CH₂)_{q}—N(R⁵)—S(O)₂R⁸, —CH₂—S(O)₂N(R⁵)R⁶, —C₁₋₆ haloalkyl, —OCF₃, —OCH(F)₂, —OCH₂F, —C(O)OR⁵, —OR⁵, —R⁸CN, CN, —SO₂R⁹; —(CH₂)ₙheteroaryl, —(CH₂)ₙheterocycyl, —(CH₂)ₙcycloalkyl, —(CH₂)ₙcycloalkenyl, and —(CH₂)ₙaryl;
R² represents a group selected from hydrogen, C₁₋₁₀alkyl, C₂₋₁₀alkenyl, C₂₋₁₀alkynyl, cycloalkyl, cycloalkenyl heterocyclyl, aryl, and heteroaryl, each of which may be optionally substituted by one or more of: C₁₋₁₀alkyl, C₂₋₁₀alkenyl, C₂₋₁₀alkynyl, cycloalkyl, heterocyclyl, aryl, heteroaryl, C₁₋₆ haloalkyl, halogen, —CN, —OR⁴, —(CH₂)ₙCOR⁴, —CO₂R⁴, —OCOR⁴, —(CH₂)ₙNR⁵R⁶, —(NH)ₚCONR⁵R⁶, —OCONR⁵R⁷, and —NHC(O)OR⁷;
R³ represents a group selected from halogen and CN;
R⁴ represents a group selected from hydrogen, C₁₋₆ alkyl, C₂₋₆alkenyl, C₂₋₆alkynyl, —(CH₂)ₙcycloalkyl, —(CH₂)ₙcycloalkenyl, —(CH₂)ₙheterocyclyl, —(CH₂)ₙaryl, and —(CH₂)ₙheteroaryl;
R⁵ and R⁶ are independently selected from hydrogen and C₁₋₄alkyl;
R⁷ represents a group selected from H, C₁₋₆alkyl, C₂₋₆ alkenyl, C₂₋₆alkynyl, —(CH₂)ₜcycloalkyl, —(CH₂)ₙcycloalkenyl, —(CH₂)ₜheterocyclyl, —(CH₂)ₜaryl, and —(CH₂)ₜheteroaryl;
R⁸ represents a group selected from C₁₋₄alkyl;
m represents an integer selected from 1, 2, 3, 4 and 5;
n represents an integer selected from 0, 1, 2, 3, 4 and 5;
t represents an integer selected from 1 and 2;
p represents an integer selected from 0 and 1;
q represents an integer selected from 0, 1 and 2.

The compound(s) are believed to be of use in the treatment of diseases where under-activation of the HM74A receptor contributes to the disease or where activation of the receptor will be beneficial. For example in treatment of diseases of lipid metabolism including dyslipidaemia and hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia. As such, the compounds may also find favour as therapeutics for coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity. As such the compounds of the present invention may find use as agonists or partial agonists of HM74A. The compound(s) may also be of use in the treatment of inflammatory diseases or conditions, as set out further below.

In one embodiment, R¹ represents optionally substituted —(CH₂)_{q}-aryl or optionally substituted —(CH₂)_{q}-heteroaryl. In a further embodiment R¹ represents —(CH₂)_{q}-aryl for example —(CH₂)_{q}-phenyl, or —(CH₂)_{q}-heteroaryl for example —(CH₂)_{q}-imidazoyl, and which in a further embodiment is substituted by halogen, for example F or Cl. In a further embodiment, R¹ represents optionally substituted —(CH₂)_{q}-aryl. In a further embodiment R¹ represents —(CH₂)_{q} aryl and may be substituted by a group selected from C₁₋₆alkyl, C₂₋₆alkenyl, C₂₋₆alkynyl, halogen, —NH₂, —OCF₃, —OCH(F)₂, —OCH₂F, —R⁸CN, CN and —SO₂R⁹. In one embodiment R¹ represents —(CH₂)_{q}-aryl which is not further substituted.

In another embodiment, R¹ represents —(CH₂)_{q}-aryl wherein q is 0, and which in a further embodiment is substituted by a group selected from —OR⁵ for example —OH or OCH₃, halogen, for example F or Cl, —OCF₃, C₁₋₆haloalkyl for example —CF₃. In a further embodiment R¹ represents —(CH₂)_{q}aryl for example (CH₂)₂phenyl and —(CH₂)_{q}heteroaryl for example (CH₂)₄imidazolyl. In a further embodiment R¹ represents —(CH₂)_{q}-aryl wherein q is 0 and which may be substituted by a group selected from —OR⁵ for example —OH or OCH₃. In a further embodiment R¹ represents —(CH₂)_{q}-aryl which is not further substituted, wherein q is 0.

In another embodiment R¹ represents —(CH₂)ₙaryl for example (CH₂)₂phenyl and —(CH₂)ₙheteroaryl for example (CH₂)₄imidazolyl wherein q is 1 or 2. In a further embodiment R¹ represents —(CH₂)_{q}-aryl wherein q is 1 or 2, and which in a further embodiment is substituted by a group selected from C₁₋₆alkyl, C₂₋₆alkenyl, C₂₋₆alkynyl, halogen, —NH₂, —OCF₃, —OCH(F)₂, —OCH₂F, —R⁸CN, CN and —SO₂R⁹. In a further embodiment R¹ represents —(CH₂)_{q}-aryl wherein q is 1 or 2, and which is substituted by halogen, for example F or Cl, —OCF₃. In one embodiment R¹ represents —(CH₂)_{q}-aryl which is not further substituted, wherein q is 1 or 2.

In one embodiment R² represents C₁₋₁₀ alkyl which may be optionally substituted by one or more of: cycloalkyl, heterocyclyl, aryl, heteroaryl, C₁₋₆haloalkyl, halogen, —CN, —OR⁴, —(CH₂)ₙCOR⁵, —C(O)OR⁴, —OCOR⁴, —(CH₂)ₙNR⁵R⁶, and —(NH)ₚCONR⁵R⁶. In a further embodiment R² represents C₁₋₁₀ alkyl which may be optionally substituted by one or more of: cycloalkyl, C₁₋₆haloalkyl, halogen, —CN, —OR⁴, —(CH₂)ₙCOR⁵, —C(O)OR⁴, —OCOR⁴, —(CH₂)ₙNR⁵R⁶, and —(NH)ₚCONR⁵R⁶. In a further embodiment R² represents C₁₋₁₀ alkyl which is not further substituted. In a further embodiment, R² is selected from C₃₋₆ alkyl, for example butyl or pentyl, for example n-butyl or n-pentyl.

In one embodiment, R³ represents halogen. In a further embodiment, R³ is selected from chlorine and bromine. In a further embodiment, R³ represents chlorine.

In one embodiment, R⁵ represents hydrogen or methyl. In a further embodiment, R⁵ represents hydrogen.

According to one aspect of this invention we provide at least one chemical entity selected from compounds of formula (Ia)

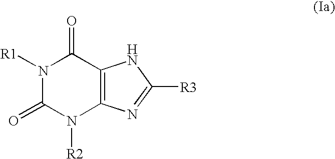

and pharmaceutically acceptable derivatives thereof, wherein
R¹ represents a group selected from
—(CH₂)_{q}-cycloalkenyl, —(CH₂)_{q}-aryl and —(CH₂)_{q}-heteroaryl;
Wherein if q is an integer selected from 1 or 2, the R¹ ring may be substituted by one or more groups independently selected from
C₁₋₆alkyl, C₂₋₆alkenyl, C₂₋₆alkynyl, halogen, —NH₂, —(CH₂)_{q}—(O)ₚ—(CH₂)_{q}—N(R⁵)C(O)OR⁸, —(CH₂)_{q}—N(R⁵)C(O)R⁸, —(CH₂)_{q}(O)ₚ—(CH₂)_{q}—C(O)NR⁵R⁶, —(CH₂)_{q}—N(R⁵)C(O)N(R⁵)R⁶, —(CH₂)_{q}—C(O)N((CH₂)ₘOH)R⁵, —(CH₂)_{q}—N(R⁵)—S(O)₂R⁸, —CH₂—S(O)₂N(R⁵)R⁶, —C₁₋₆haloalkyl, —OCF₃, —OCH(F)₂, —OCH₂F, —C(O)OR⁵, —OR⁵, —(R⁸)ₚCN, and —SO₂R⁹;
Wherein if q is 0, the R¹ ring may be substituted by one or more groups independently selected from C₁₋₆alkyl, C₂₋₆alkenyl, C₂₋₆alkynyl, halogen, —NH₂, —(CH₂)_{q}—(O)ₚ—(CH₂)_{q}—N(R⁵)C(O)OR⁸, —(CH₂)_{q}—N(R⁵)C(O)R⁸, —(CH₂)_{q}—(O)ₚ—(CH₂)_{q}—C(O)NR⁵R⁶, —(CH₂)_{q}—N(R⁵)C(O)N(R⁵)R⁶, —(CH₂)_{q}—C(O)N((CH₂)ₘOH)R⁵, —(CH₂)_{q}—N(R⁵)—S(O)₂R⁸, —CH₂—S(O)₂N(R⁵)R⁶, —C₁₋₆ haloalkyl, —OCF₃, —OCH(F)₂, —OCH₂F, —C(O)OR⁵, —OR⁵, —(R⁸)ₚCN, —SO₂R⁹; —(CH₂)ₙheteroaryl, —(CH₂)ₙheterocycyl, —(CH₂)ₙcycloalkyl, —(CH₂)ₙcycloalkenyl, and —(CH₂)ₙaryl;
R² represents a group selected from hydrogen, C₁₋₁₀alkyl, C₂₋₁₀alkenyl, C₂₋₁₀alkynyl, cycloalkyl, cycloalkenyl heterocyclyl, aryl, and heteroaryl, each of which may be optionally substituted by one or more of: C₁₋₁₀alkyl, C₂₋₁₀alkenyl, C₂₋₁₀alkynyl, cycloalkyl, heterocyclyl, aryl, heteroaryl, C₁₋₆ haloalkyl, halogen, —CN, —OR⁴, —(CH₂)ₙCOR⁴, —CO₂R⁴, —OCOR⁴, —(CH₂)ₙNR⁵R⁶, —(NH)ₚCONR⁵R⁶, —OCONR⁵R⁷, and —NHC(O)OR⁷;
R³ represents a group selected from halogen and CN;
R⁴ represents a group selected from H, C₁₋₆ alkyl, C₂₋₆alkenyl, C₂₋₆alkynyl, —(CH₂)ₙcycloalkyl, —(CH₂)ₙcycloalkenyl, —(CH₂)ₙheterocyclyl, —(CH₂)ₙaryl, and —(CH₂)ₙheteroaryl;
R⁵ and R⁶ are independently selected from hydrogen and C₁₋₄alkyl;
R⁷ represents a group selected from H, C₁₋₆alkyl, C₂₋₆ alkenyl, C₂₋₆alkynyl, —(CH₂)ₜcycloalkyl, —(CH₂)ₙcycloalkenyl, —(CH₂)ₜheterocycyl, —(CH₂)ₜaryl, and —(CH₂)ₜheteroaryl;
R⁸ represents a group selected from C₁₋₄alkyl;
m represents an integer selected from 1, 2, 3, 4 and 5;
n represents an integer selected from 0, 1, 2, 3, 4 and 5;
t represents an integer selected from 1 and 2;
p represents an integer selected from 0 and 1;
q represents an integer selected from 0, 1 and 2.

In one embodiment for formula (Ia) R¹ represents optionally substituted —(CH₂)_{q}-aryl or optionally substituted —(CH₂)_{q}-heteroaryl. In a further embodiment, R¹ represents —(CH₂)_{q}aryl for example —(CH₂)_{q}-phenyl, or —(CH₂)_{q}-heteroaryl for example —(CH₂)_{q}-imidazoyl wherein q is selected from 1 and 2, and which in a further embodiment is substituted by a group selected from —OR⁵ for example —OH or OCH₃, halogen, for example F or Cl, —OCF₃, and C₁₋₆ haloalkyl for example —CF₃.

In one embodiment, the compound of formula (I) or (Ia) is other than 1H-purine-2,6-dione, 8-bromo-1-[(4-chlorophenyl)methyl]-3,7-dihydro-3-(2-methylphenyl).

With regard to stereoisomers, the compounds of formula (I) may have one or more asymmetric carbon atom and may occur as recemates, racemic mixtures and as individual enantiomers or diastereomers. All such isomeric forms are included within the present invention, including mixtures thereof.

Where a compound of formula (I) or (Ia) contains an alkenyl or alkenylene group, cis (E) and trans (Z) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, e.g. by fractional crystallisation, chromatography or HPLC of a stereoisomeric mixture of the agent may also be prepared from a corresponding optically pure intermediate or by resolution, such as HPLC of the corresponding racemate using a suitable chiral support or by fractional crystallisation of the diastereoisomeric salts formed by reaction of the corresponding racemate with a suitable optically active acid or base, as appropriate.

Furthermore, some of the crystalline forms of the compounds of formula (I) or (Ia) may exist as polymorphs, which are included in the present invention. One form may have an advantage over another form, for example one form may have improved stability over another form.

It is to be understood that the present invention includes any combination of particular embodiments and covers all combinations of particular substituents described hereinabove.

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

As used herein, the term “alkyl” (when used as a group or as part of a group) refers to a straight or branched hydrocarbon chain unless specified otherwise, containing the specified number of carbon atoms. For example, C₃-C₁₀alkyl means a straight or branched hydrocarbon chain containing at least 3 and at most 10 carbon atoms. Examples of alkyl as used herein include, but are not limited to methyl (Me), ethyl (Et), n-propyl and i-propyl.

The term “alkenyl” as used herein refers to a straight or branched hydrocarbon chain containing the specified number of carbon atoms which contains one or more double bonds.

The term “alkynyl” as used herein refers to a straight or branched hydrocarbon chain containing the specified number of carbon atoms which contains one or more triple bonds.

The term “cycloalkyl” as used herein refers to a saturated monocytic hydrocarbon ring of 3 to 8 carbon atoms. Examples of such groups include cyclopropyl, cyclobutyl, cyclopentyl, cyclohexyl, cycloheptyl or cyclooctyl and the like.

The term “aryl” as used herein refers to a C₉ monocyclic or bicyclic hydrocarbon ring wherein at least one ring is aromatic. Examples of such groups include phenyl and the like.

The term “heteroaryl” as used herein refers to a 5-6 membered monocyclic aromatic or a fused 8-10 membered bicyclic aromatic ring containing 1 to 4 heteroatoms selected from oxygen, nitrogen and sulphur. Examples of such monocyclic aromatic rings include thienyl, furyl, furazanyl, pyrrolyl, triazolyl, tetrazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, tetrazinyl and the like. Examples of such fused aromatic rings include quinolinyl, isoquinolinyl, quinazolinyl, quinoxalinyl, pteridinyl, cinnolinyl, phthalazinyl, naphthyridinyl, indolyl, isoindolyl, azaindolyl, indolizinyl, indazolyl, purinyl, pyrrolopyridinyl, furopyridinyl, benzofuranyl, isobenzofuranyl, benzothienyl, benzoimidazolyl, benzoxazolyl, benzoisoxazolyl, benzothiazolyl, benzoisothiazolyl, benzoxadiazolyl, benzothiadiazolyl and the like.

The term “cycloalkenyl” as used herein refers to an unsaturated non-aromatic monocyclic hydrocarbon ring of 3 to 8 carbon atoms containing one or more carbon-carbon double bonds. Examples of such groups include cyclopropenyl, cyclobutenyl, cyclopentenyl, cyclohexenyl, cycloheptenyl, cyclooctenyl and the like.

The term ‘heterocyclyl’ as used herein refers to a 4-7 membered monocyclic ring or a fused 8-12 membered bicyclic ring which may be saturated or partially unsaturated containing 1 to 4 heteroatoms selected from oxygen, nitrogen or sulphur. There may be one or more optional oxo substituents on the ring carbon atoms. Examples of such monocyclic rings include pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, diazepanyl, azepanyl and the like. Examples of such bicyclic rings include indolinyl, isoindolinyl, benzopyranyl, quinuclidinyl, 2,3,4,5-tetrahydro-1H-3-benzazepine, tetrahydroisoquinolinyl and the like.

The terms “halogen” or “halo” as used herein refer to fluorine, chlorine, bromine and iodine.

The term ‘C₁₋₆ haloalkyl’ as used herein refers to a C₁₋₆ alkyl group as defined herein wherein at least one hydrogen atom is replaced with halogen. Examples of such groups include fluoroethyl, trifluoromethyl or trifluoroethyl and the like.

As used herein, where a group is referred to as being “substituted” by another group or having “one or more substituents” unless a particular position for such a substitution is specified it is to be understood that a substitution may be present at any position in the group.

The term “pharmaceutically acceptable derivative” as used herein refers to any pharmaceutically acceptable derivative of a compound of the present invention, for example, salts, solvates or esters, which upon administration to a mammal, such as a human, is capable of providing (directly or indirectly) such a compound or an active metabolite thereof. Such derivatives are clear to those skilled in the art, without undue experimentation, and with reference to the teaching of Burger's Medicinal Chemistry And Drug Discovery, 5th Edition, Vol 1: Principles And Practice, which is incorporated herein by reference.

As used herein, the term “pharmaceutically acceptable” used in relation to an ingredient (active ingredient, diluent, excipient or carrier) which may be included in a pharmaceutical formulation for administration to a patient, refers to that ingredient being acceptable in the sense of being compatible with any other ingredients present in the pharmaceutical formulation and not being deleterious to the recipient thereof.

As used herein, the term “solvate” refers to a complex of variable stoichiometry formed by a solute (in this invention, a compound of formula (I) or (Ia) or a pharmaceutically acceptable derivative thereof) and a solvent. Such solvents for the purposes of the present invention may not interfere with the biological activity of the solute. The solvent used may be a pharmaceutically acceptable solvent. Examples of suitable pharmaceutically acceptable solvents include water, ethanol and acetic acid. An example of a solvent that may be used is water, in which case the solvate may be referred to as a hydrate of the solute in question.

It will be appreciated that, for pharmaceutical use, the “salt or solvate” referred to above will be a pharmaceutically acceptable salt or solvate. However, other salts or solvates may find use, for example, in the preparation of a compound of formula (I) or (Ia) or in the preparation of a pharmaceutically acceptable salt or solvate thereof.

Pharmaceutically acceptable salts include those described by Berge, Bighley and Monkhouse, J. Pharm. Sci., 1977, 66, 1-19. Suitable pharmaceutically acceptable salts include alkali metal salts formed from the addition of alkali metal bases such as alkali metal hydroxides. Examples of suitable alkali metal salts include sodium salts and potassium salts. Other suitable pharmaceutically acceptable salts include alkaline earth metal salts such as calcium salts and magnesium salts, ammonium salts; or salts with organic bases such as ethanolamine, triethanolamine, ethylene diamine, triethylmine, choline and meglumine; or salts with amino acids such as arginine, lysine and histidine.

Esters may be active in their own right and/or be hydrolysable under in vivo conditions in the human body. Suitable pharmaceutically acceptable in vivo hydrolysable ester groups include those which break down readily in the human body to leave the parent acid or its salt. An ester may be formed at a carboxylic acid (—C(O)OH) group, by methods well known in the art involving reaction with the corresponding alcohol. For example, esters may be C₁₋₆alkyl esters, e.g. methyl esters, ethyl esters, and the like.

As used herein, the term “compounds of the invention” means the compounds according to Formula I and the pharmaceutically acceptable derivatives thereof. The term “a compound of the invention” means any one of the compounds of the invention as defined above.

As used herein the term “at least one chemical entity” means at least one chemical substance chosen from the group of compounds consisting of compounds of formula I and pharmaceutically acceptable derivatives thereof.

Compounds of formula (I) or (Ia) are of potential therapeutic benefit in the treatment and amelioration of the symptoms of many diseases of lipid metabolism including dyslipidaemia and hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa, obesity. As such, the compounds may also find favour as therapeutics for coronary artery disease, thrombosis, angina, chronic renal failure, peripheral vascular disease and stroke.

It has been reported that the HM74 and HM74A receptors are involved in inflammation (WO02084298). Inflammation represents a group of vascular, cellular and neurological responses to trauma. Inflammation can be characterised as the movement of inflammatory cells such as monocytes, neutrophils and granulocytes into the tissues. This is usually associated with reduced endothelial barrier function and oedema into the tissues. Inflammation with regards to disease typically is referred to as chronic inflammation. Such chronic inflammation may manifest itself through disease symptoms. The aim of anti-inflammatory therapy is therefore to reduce this chronic inflammation and allow for the physiological process of healing and tissue repair to progress.

Examples of inflammatory diseases or conditions for which the compounds of the present invention may demonstrate utility include those of the joint, for example arthritis (e.g. rheumatoid arthritis, osteoarthritis, prosthetic joint failure), or the gastrointestinal tract (e.g. ulcerative colitis, Crohn's disease, and other inflammatory bowel and gastrointestinal diseases, gastritis and mucosal inflammation resulting from infection, the enteropathy provoked by non-steroidal anti-inflammatory drugs), of the lung (e.g. adult respiratory distress syndrome, asthma, cystic fibrosis, or chronic obstructive pulmonary disease), of the heart (e.g. myocarditis), of nervous tissue (e.g. multiple sclerosis), of the pancreas, (e.g. inflammation associated with diabetes mellitus and complications thereof, of the kidney (e.g. glomerulonephritis), of the skin (e.g. dermatitis, psoriasis, eczema, urticaria, burn injury), of the eye (e.g. glaucoma) as well as of transplanted organs (e.g. rejection) and multi-organ diseases (e.g. systemic lupus erythematosis, sepsis) and inflammatory sequelae of viral or bacterial infections and inflammatory conditions associated with atherosclerosis and following hypoxic or ischaemic insults (with or without reperfusion), for example in the brain or in ischaemic heart disease.

In one embodiment, the compounds of this invention are useful in the treatment and prevention of inflammation, diabetes and cardiovascular diseases or conditions including atherosclerosis, arteriosclerosis, hypertriglyceridemia, and mixed dyslipidaemia.

Nicotinic acid has a significant side effect profile, possibly because it is dosed at high level (gram quantities daily). The most common side effect is an intense cutaneous flushing. In certain embodiments of the present invention the compounds may exhibit reduced side effects compared to nicotinic acid. HM74A has been identified as a high affinity receptor for nicotinic acid whilst HM74 is a lower affinity receptor. The compounds of the present invention show greater affinity for HM74A than for HM74 therefore may find use as selective HM74A agonists or partial agonists.

The potential for compounds of formula (I) or (Ia) to activate HM74A may be demonstrated, for example, using the following in vitro whole cell assays:

### In-Vitro Testing

For transient transfections, HEK293T cells (HEK293 cells stably expressing the SV40 large T-antigen) were maintained in DMEM containing 10% foetal calf serum and 2 mM glutamine. Cells were seeded in 90 mm culture dishes and grown to 60-80% confluence (18-24 h) prior to transfection. Human HM74A (GenBank™ accession number AY148884) was subclone in to a mammalian expression vector (pcDNA3; Invitrogen) and transfected using Lipofectamine reagent. For transfection, 9 μg of DNA was mixed with 30 μl Lipofectamine in 0.6 ml or Opti-MEM (Life Technologies Inc.) and was incubated at room temperature for 30 min prior to the addition of 1.6 ml of Opti-MEM. Cells were exposed to the Lipofectamine/DNA mixture for 5 h and 6 ml of 20% (v/v) foetal calf serum in DMEM was then added. Cells were harvested 48 h after transfection. Pertussis toxin treatment was carried out by supplementation into media at 50 ngml⁻¹ for 16 h. All transient transfection studies involved co-transfection of receptor together with the G_{i/o} G protein, Gₒ₁α.

For generation of stable cell lines the above method was used to transfect CHO-K1 cells seeded in six well dishes grown to 30% confluence. These cells were maintained in DMEM F-12 HAM media containing 10% foetal calf serum and 2 mM glutamine. 48 h post-transfection the media was supplemented with 400 μg/ml Geneticin (G418, Gibco) for selection of antibiotic resistant cells. Clonal CHO-K1 cell lines stably expressing HM74A were confirmed by [³⁵S]-GTPγS binding measurements, following the addition of nicotinic acid.

P2 membrane preparation—Plasma membrane-containing P2 particulate fractions were prepared from cell pastes frozen at −80° C. after harvest. All procedures were carried out at 4° C. Cell pellets were resuspended in 1 ml of 10 mM Tris-HCl and 0.1 mM EDTA, pH 7.5 (buffer A) and by homogenisation for 20 s with a Ultra Turrax followed by passage (5 times) through a 25-gauge needle. Cell lysates were centrifuged at 1,000 g for 10 min in a microcentrifuge to pellet the nuclei and unbroken cells and P2 particulate fractions were recovered by microcentrifugation at 16,000 g for 30 min. P2 particulate fractions were resuspended in buffer A and stored at −80° C. until required.

[³⁵S]-GTPγS binding—assays were performed at room temperature in 384-well format based on methods described previously, (Wieland, T. and Jakobs, K. H. (1994) Methods Enzymol. 237, 3-13). Briefly, the dilution of standard or test compounds were prepared and added to a 384-well plate in a volume of 10 μl. Membranes (HM74A or HM74) were diluted in assay buffer (20 mM HEPES, 100 mM NaCl, 10 mM MgCl₂, pH7.4) supplemented with saponin (60 μg/ml), Leadseeker WGA beads (Amersham; 250 μg/well) and 10 μM GDP, so that the 20 μl volume added to each well contains 5 μg of membranes. [³⁵S]-GTPγS (1170 Ci/mmol, Amersham) was diluted (1:1500) in assay buffer and 20 μl added to each well. Following the addition of the radioligand, the plates were sealed, pulse spun and incubated for 4 hours at room temperature. At the end of the incubation period the plates were read on a Leadseeker machine (VIEWLUX PLUS; Perkin-Elmer) to determine the levels of specific binding.

These assays were refined by reducing the final assay volume to 10 μl. For this 10 μl assay a revised protocol was used. This involved the use of only 100 nl of standard or test compound per well of a 384-well plate and 1.5 μg membrane and 100 μg Leadseeker WGA beads. For the low volume protocol, membrane, beads and [³⁵S]-GTPγS were mixed together and then 10 μl of this mix were dispensed to each well. Incubation and plate read were identical for the 10 μl and 50 μl assays.

All exemplified compounds were tested in one or both of the [³⁵S]-GTPγS binding assays described above (i.e. the 10 μl and 50 μl assays).

Data was analysed by curve fitting as carried out using a Four Parameter Logistical equation using the XC50 software package (max 2 points deleted from any one curve). Specific binding is expressed as pEC₅₀ and as % efficacy compared to the maximal response of nicotinic acid binding.

### In-Vivo Testing

HM74A agonists can be tested in male Spague-Dawley rats (200-250 g) which have been fasted for at least 12 hours prior to the study. The compounds are dosed intravenously at either 1 or 3 mg/kg (5 ml/kg) or by oral gavage at doses ranging from 1-30 mg/kg (10 ml/kg). Blood samples (0.3 ml tail vein bleed) can be taken pre-dose and at three times post-dose (times ranging from 15 minutes to 6 hours post-dose). Each blood sample is transferred to a heparin tube (Becton Dickinson Microtainer, PST LH) and centrifuged (10,000 g for 5 minutes) to produce a plasma sample. The plasma samples are assayed for levels of non-esterified fatty acids (NEFA) using a commercially available kit (Randox). Inhibition of plasma NEFA levels, relative to pre-dose levels, are used as a surrogate for HM74A agonist activity.

In order to determine whether HM74A compounds exhibit the flushing response associated with nicotinic acid they can be dosed to conscious guinea-pigs. Male Dunkin Hartley guinea pigs (300-600 g; n=10-20 per group) are fasted for at least 12 hours, but not in excess of 24 hours prior to experimention. Pre-study blood samples (0.5 ml) are taken from each animal by cardiac puncture under recovery anaesthesia (Isoflurane 3.5% with additional O2 (1 L/min)). Ear temperature measurements are taken by placing the left ear of each animal over an infra-red temperature probe. Measurements are taken at one minute intervals from 5 minutes pre-dose to 30 minutes post-dose. Temperature measurements are then taken at 15 minute intervals up to 2 hours post-dose. Animals receive test compounds by oral gavage (5 ml/kg). Blood samples (0.5 ml) are taken by cardiac puncture under terminal anaesthesia. Blood samples are taken from individual animals to provide data at 0.5, 1, 2, 3, and 4 hours post-dose. All blood samples are placed on a blood roller for 5 minutes then stored on ice until the end of the study. Following centrifugation (12000 g for 5 min) the plasma is transferred into fresh tubes and stored at −20° C. until assayed for NEFA concentrations.

Compounds according to Formula (I) or (Ia) have been synthesised (see synthetic examples below) and tested in the [³⁵S]-GTPγS binding assays discussed above.

All exemplified compounds have a pEC₅₀ of 4.3 (+/−0.3 log unit) or greater and an efficacy of 30% or greater (in relation to nicotinic acid) in the [³⁵S]-GTPγS binding assays described above, in which they were tested.

### General Purification and Analytical Methods:

### LC/MS: Method

Analytical HPLC was conducted on a Supelcosil™ ABZ+PLUS column (Supelco) (3 μm, 3.3 cm×4.6 mm ID) eluting with 0.1% HCO₂H and 0.01 M ammonium acetate in water (solvent A), and 95% MeCN and 5% water (containing 0.5% HCO₂H) (solvent B), using the following elution gradient 0-0.7 min 0% B, 0.7-4.2 min 0→100% B, 4.2-4.6 minutes 100% B, 4.6-4.8 min 100→0% B at a flow rate of 3 ml/min. The diode array UV detection was carried out in the range 215 to 330 nm. The mass spectra (MS) were recorded on a Waters ZQ mass spectrometer using electrospray positive ionisation [(ES+ve to give MH⁺ and M(NH₄)⁺ molecular ions] or electrospray negative ionisation [(ES-ve to give (M-H)⁻ molecular ion] modes. Only the parent ion of major isotopes quoted.

¹H NMR spectra were recorded using a Bruker DPX 400 MHz spectrometer using tetramethylsilane as the standard.

Biotage™ chromatography refers to purification carried out using either the Flash 40i or Flash 150i purification systems, sold by Biotage AB, using cartridges pre-packed with KPSil (silica).

Companion™ system refers to the Teledyne Isco Combiflash Companion™ purification system. This is a gradient controlled purification system with integral, variable wavelength UV detection with the capability to trigger automated fraction collection by UV threshold.

Mass directed autoprep (MDAP) refers to methods where the material was purified by high performance liquid chromatography using either a Supelcosil™ ABZ+5 μm column (10 cm×20 mm i.d.) or a Supelcosil™ ABZ+10 μm column (15 cm×30 mm i.d.) with a suitable gradient of solvent A: 0.1% HCO₂H in water and solvent B: 95% MeCN, 5% water (containing 0.5% HCO₂H). The Waters 2767 inject/collector was triggered by a MicroMass ZQ Mass Spectrometer on detecting the mass of interest (using Micromass MassLynx software).

Preparative HPLC (Autoprep HPLC or Autoprep) refers to methods where the material was purified by high performance liquid chromatography on a Supelcosil™ ABZ+5 μm column (10 cm×21.2 mm i.d.) with a suitable gradient of 0.1% HCO₂H in water and MeCN (with 0.5% HCO₂H). The Gilson 233 fraction collector was triggered by UV detection.

SPE (solid phase extraction) refers to the use of polyethylene cartridges which are pre-packed with sorbent used for purification. The sorbent contained in these cartridges will be specified. Examples used are detailed below:

C18 SPE refers to the use of cartridges pre-packed with 40 μM C18 funtionalised silica sorbent (sold by Varian Inc.). The compound was loaded, typically in 50:50 DMSO/MeOH, onto a cartridge previously conditioned with MeCN and equilibrated with 5% MeCN in water. The product was eluted with a suitable gradient of 0.1% HCO₂H in water and MeCN (0.5% HCO₂H).

Aminopropyl SPE or column refers to the use of cartridges pre-packed with 40 μm-120 μm aminopropyl functionalized silica (sold by Varian Inc.). The crude product is typically loaded in DCM/MeOH mixtures onto a cartridge previously conditioned with MeOH. The neutral components were eluted with MeOH and/or DCM (3 or 4 column volumes) and the acidic components usually eluted with an eluent containing a proportion of AcOH (2-20%).

Oasis™ Cartridges/Oasis™ SPE's refer to SPE cartridges packed with a polymeric sorbent manufactured by the Waters Corporation. These are typically conditioned with 3 column volumes of MeOH and equilibrated with water before the sample is loaded. Salts and inorganics are eluted with water and the product typically eluted with MeOH or MeCN.

GreenHouse™ refers to a 24 reaction parallel synthesiser platform available from RDT Ltd, UK

As indicated above, compounds of Formula (I) or (Ia) may find use in human or veterinary medicine, for example as activators of HM74A, in the management of dyslipidaemia and hyperlipoproteinaemia.

Thus, there is provided as another embodiment of the present invention at least one compound of formula (I) or (Ia) or a pharmaceutically acceptable derivative thereof, for use in human or veterinary medicine, for example in the treatment of disorders of lipid metabolism including dyslipidaemia and hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa and obesity. As such, the compounds are also provided for use in the treatment of coronary artery disease, thrombosis, angina, chronic renal failure, peripheral vascular disease and stroke.

There is provided as a further embodiment of the present invention at least one compound of formula (I) or (Ia) or a pharmaceutically acceptable derivative thereof, for use in the manufacture of a medicament for the treatment of disorders of lipid metabolism including dyslipidaemia and hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia, type II diabetes mellitus, type 4-diabetes, insulin resistance, hyperlipidaemia, anorexia nervosa, obesity. As such, the compounds are also provided for use in the treatment of coronary artery disease, thrombosis, angina, chronic renal failure, peripheral vascular disease and stroke.

It will be appreciated that references herein to treatment extend to prophylaxis, prevention of recurrence and suppression of symptoms as well as the treatment of established conditions.

In one embodiment of the invention, there is provided at least one compound of formula (I) or (Ia) or a pharmaceutically acceptable derivative thereof for use in the treatment of disorders of lipid metabolism including dyslipidaemia and hyperlipoproteinaemia. For example, the use is provided of at least one compound of Formula (I) or (Ia) or a pharmaceutically acceptable derivative thereof in the treatment of diabetic dyslipidaemia, mixed dyslipidaemia, heart failure, hypercholesteraemia, type II diabetes mellitus, type I diabetes, insulin resistance, hyperlipidaemia, anorexia nervosa, obesity, coronary artery disease, thrombosis, angina, chronic renal failure, stroke and cardiovascular disease including atherosclerosis, arteriosclerosis, and hypertriglyceridaemia.

It is to be understood that this embodiment of the present invention includes any combination of particular embodiments and covers all combinations of particular substituents described herein above for compounds of Formula (I) or (Ia).

Additionally, the present invention provides the use of at least one compound of formula (I) or (Ia) or a pharmaceutically acceptable derivative thereof, in the manufacture of a medicament for the treatment of inflammatory diseases or conditions of the joint, for example, arthritis (e.g. rheumatoid arthritis, osteoarthritis, prosthetic joint failure), or of the gastrointestinal tract (e.g. ulcerative colitis, Crohn's disease, and other inflammatory bowel and gastrointestinal diseases, gastritis and mucosal inflammation resulting from infection, the enteropathy provoked by non-steroidal anti-inflammatory drugs), of the lung (e.g. adult respiratory distress syndrome, asthma, cystic fibrosis, or chronic obstructive pulmonary disease), of the heart (e.g. myocarditis), of nervous tissue (e.g. multiple sclerosis), of the pancreas, (e.g. inflammation associated with diabetes mellitus and complications thereof, of the kidney (e.g. glomerulonephritis), of the skin (e.g. dermatitis, psoriasis, eczema, urticaria, burn injury), of the eye (e.g. glaucoma) as well as of transplanted organs (e.g. rejection) and multi-organ diseases (e.g. systemic lupus erythematosis, sepsis) and inflammatory sequelae of viral or bacterial infections and inflammatory conditions associated with atherosclerosis and following hypoxic or ischaemic insults (with or without reperfusion), for example in the brain or in ischaemic heart disease.

In a further or alternative embodiments there is provided a method for the treatment of a human or animal subject with a condition where under-activation of the HM74A receptor contributes to the condition or where activation of the receptor will be beneficial, which method comprises administering to said human or animal subject an effective amount of at least one compound of formula (I) or (Ia) or a pharmaceutically acceptable derivative thereof.

Again, it to be understood that this embodiment of the present invention includes any combination of particular embodiments and covers all combinations of particular substituents described herein above for compounds of Formula (I) or (Ia).

In one embodiment, the present invention provides a method for the treatment of disorders of lipid metabolism including dyslipidaemia and hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa and obesity, which method comprises administering to said human or animal subject an effective amount of at least one compound of formula (I) or (Ia) or a pharmaceutically acceptable derivative thereof. As such, these compounds may also find favour in methods for the treatment of coronary artery disease, thrombosis, angina, chronic renal failure, peripheral vascular disease and stroke, which methods comprise administering to said human or animal subject an effective amount of at least one compound of formula (I) or (Ia) or a pharmaceutically acceptable derivative thereof.

The amount of a compound of formula (I) or (Ia) or a pharmaceutically acceptable derivative thereof which is required to achieve the desired biological effect will, of course, depend on a number of factors, for example, the mode of administration and the precise clinical condition of the recipient. In general, the daily dose will be in the range of 0.1 mg-1 g/kg, typically 0.1-100 mg/kg. An intravenous dose may, for example, be in the range of 0.01 mg to 0.1 g/kg, typically 0.01 mg to 10 mg/kg, which may conveniently be administered as an infusion of from 0.1 μg to 1 mg, per minute. Infusion fluids suitable for this purpose may contain, for example, from 0.01 μg to 0.1 mg, per millilitre. Unit doses may contain, for example, from 0.01 μg to 1 g of a compound of the invention. Thus ampoules for injection may contain, for example, from 0.01 μg to 0.1 g and orally administrable unit dose formulations, such as tablets or capsules, may contain, for example, from 0.1 mg to 1 g, for example from 5 mg to 50 mg.

A compound of formula (I) or (Ia) or a pharmaceutically acceptable derivative thereof may be employed as the compound per se in the treatment of a disease where under-activation of the HM74A receptor contributes to the disease or where activation of the receptor will be beneficial, an example of this is where a compound of the present invention is presented with an acceptable carrier in the form of a pharmaceutical formulation. The carrier must, of course, be acceptable in the sense of being compatible with the other ingredients of the formulation and must not be deleterious to the recipient. The carrier may be a solid or a liquid, or both, and may be formulated with the compound of the invention as a unit-dose formulation, for example, a tablet, which may contain from 0.05% to 95% by weight of the compound of the invention.

The formulations include those suitable for oral, rectal, topical, buccal (e.g. sub-lingual) and parenteral (e.g. subcutaneous, intramuscular, intradermal or intravenous) administration.

There is also provided according to the invention a process for preparation of such a pharmaceutical composition which comprises mixing the ingredients.

Formulations suitable for oral administration may be presented in discrete units, such as capsules, cachets, lozenges or tablets, each containing a predetermined amount of a a compound of formula (I) or (Ia) or a pharmaceutically acceptable derivative thereof; as a powder or granules; as a solution or a suspension in an aqueous or non-aqueous liquid; or as an oil-in-water or water-in-oil emulsion. In general, the formulations are prepared by uniformly and intimately admixing the compound of formula (I) or (Ia) or a pharmaceutically acceptable derivative thereof, with a liquid or finely divided solid carrier, or both, and then, if necessary, shaping the product. For example, a tablet may be prepared by compressing or moulding a powder or granules of the compound of formula (I) or (Ia) or a pharmaceutically acceptable derivative thereof optionally with one or more accessory ingredients. Compressed tablets may be prepared by compressing, in a suitable machine, the compound in a free-flowing form, such as a powder or granules optionally mixed with a binder, lubricant, inert diluent and/or surface active/dispersing agent(s). Moulded tablets may be made by moulding, in a suitable machine, the powdered compound moistened with an inert liquid diluent.

Tablets and capsules for oral administration may contain conventional excipients such as binding agents, for example syrup, acacia, gelatin, sorbitol, tragacanth, mucilage of starch or polyvinyl pyrrolidone; fillers, for example, lactose, microcrystalline cellulose, sugar, maize-starch, calcium phosphate or sorbitol; lubricants, for example, magnesium stearate, stearic acid, talc, polyethylene glycol or silica; disintegrants, for example, potato starch, croscarmellose sodium or sodium starch glycollate; or wetting agents such as sodium lauryl sulphate. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxymethyl cellulose, hydroxypropylmethyl cellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example almond oil, fractionated coconut oil, oily esters, propylene glycol or ethyl alcohol; or preservatives, for example, methyl or propyl p-hydroxybenzoates or sorbic acid. The preparations may also contain buffer salts, flavouring, colouring and/or sweetening agents (e.g. mannitol) as appropriate.

Formulations suitable for buccal (sub-lingual) administration include lozenges comprising a compound of the invention in a flavoured base, usually sucrose and acacia or tragacanth, and pastilles comprising the compound of the invention in an inert base such as gelatin and glycerin or sucrose and acacia.

Formulations of the present invention suitable for parenteral administration conveniently comprise sterile aqueous preparations of a compound of formula (I) or (Ia) or a pharmaceutically acceptable derivative thereof, the formulation may be isotonic with the blood of the intended recipient. These preparations could be administered intravenously, although administration may also be effected by means of subcutaneous, intramuscular, or intradermal injection. Such preparations may conveniently be prepared by admixing the compound of formula (I) or (Ia) or a pharmaceutically acceptable derivative thereof with water and rendering the resulting solution sterile and isotonic with the blood. Injectable compositions according to the invention will generally contain from 0.1 to 5% w/w of the compound of formula (I) or (Ia) or a pharmaceutically acceptable derivative thereof.

Thus, formulations of the present invention suitable for parenteral administration comprising a compound of formula (I) or (Ia) or a pharmaceutically acceptable derivative thereof may be formulated for parenteral administration by bolus injection or continuous infusion and may be presented in unit dose form, for instance as ampoules, vials, small volume infusions or pre-filled syringes, or in multi-dose containers with an added preservative. The compositions may take such forms as solutions, suspensions, or emulsions in aqueous or non-aqueous vehicles, and may contain formulatory agents such as anti-oxidants, buffers, antimicrobial agents and/or toxicity adjusting agents. Examples of formulations suitable for oral administration include formulations comprising a compound of formula (I) or (Ia) or a pharmaceutically acceptable derivative thereof, in 10% DMSO and 90% sodium hydrogen carbonate in sterile saline. Examples of formulations suitable for intravenous administration include formulations comprising a compound of formula (I) or (Ia) or a pharmaceutically acceptable derivative thereof, in 5% or 10% DMSO and 95% or 90% sodium hydrogen carbonate in sterile water. Alternatively, the therapeutically active agent may be in powder form for constitution with a suitable vehicle, e.g. sterile, pyrogen-free water, before use. The dry solid presentation may be prepared by filling a sterile powder aseptically into individual sterile containers or by filling a sterile solution aseptically into each container and freeze-drying.

Formulations suitable for rectal administration may be presented as unit-dose suppositories. These may be prepared by admixing a a compound of formula (I) or (Ia) or a pharmaceutically acceptable derivative thereof with one or more conventional solid carriers, for example, cocoa butter or glycerides and then shaping the resulting mixture.

Formulations suitable for topical application to the skin may take the form of an ointment, cream, lotion, paste, gel, spray, aerosol, or oil. Carriers which may be used include vaseline, lanolin, polyethylene glycols, alcohols, and combinations of two or more thereof. The compound of formula (I) or (Ia) or a pharmaceutically acceptable derivative thereof is generally present at a concentration of from 0.1 to 15% w/w of the composition, for example, from 0.5 to 2%.

By topical administration as used herein, we include administration by insufflation and inhalation. Examples of various types of preparation for topical administration include ointments, creams, lotions, powders, pessaries, sprays, aerosols, capsules or cartridges for use in an inhaler or insufflator or drops (e.g. eye or nose drops).

Ointments and creams may, for example, be formulated with an aqueous or oily base with the addition of suitable thickening and/or gelling agents and/or solvents. Such bases may thus, for example, include water and/or an oil such as liquid paraffin or a vegetable oil such as arachis oil or castor oil or a solvent such as a polyethylene glycol. Thickening agents which may be used include soft paraffin, aluminium stearate, cetostearyl alcohol, polyethylene glycols, microcrystalline wax and beeswax.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents or suspending agents.

Spray compositions may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluorethane, carbon dioxide or other suitable gas.

Capsules and cartridges for use in an inhaler or insufflator, of for example gelatin, may be formulated containing a powder mix of a compound of the invention and a suitable powder base such as lactose or starch.

The pharmaceutical compositions according to the invention may also be used in combination with other therapeutic agents, for example in combination with other classes of dyslipidaemic drugs (e.g. statins, fibrates, bile-acid binding resins or nicotinic acid).

The compounds of the instant invention may be used in combination with one or more other therapeutically active agents for example in combination with other classes of dyslipidaemic drugs e.g. 3-hydroxy-3-methylglutaryl-coenzyme A reductase inhibitors (statins) or fibrates or bile acid binding resins or nicotinic acid. The invention thus provides, in a further embodiment, the use of such a combination in the treatment of diseases where under-activation of the HM74A receptor contributes to the disease or where activation of the receptor will be beneficial and the use of at least one compound of formula (I) or (Ia) or a pharmaceutically acceptable derivative thereof in the manufacture of a medicament for the combination therapy of disorders of lipid metabolism including dyslipidaemia and hyperlipoproteinaemia such as diabetic dyslipidaemia and mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa and obesity.

When the compounds of the present invention are used in combination with other therapeutically active agents, the compounds may be administered either together or separately, sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further embodiment of the invention. The individual components of such combinations may be administered either together or separately, sequentially or simultaneously in separate or combined pharmaceutical formulations.

When combined in the same formulation it will be appreciated that the two components must be stable and compatible with each other and the other components of the formulation and may be formulated for administration. When formulated separately they may be provided in any convenient formulation, conveniently in such a manner as are known for such compounds in the art.

When in combination with a second therapeutic agent active against the same disease, the dose of each component may differ from that when the compound is used alone. Appropriate doses will be readily appreciated by those skilled in the art.

The invention thus provides, in a further embodiment, a combination comprising at least one compound of formula (I) or (Ia) or a pharmaceutically acceptable derivative thereof together with another therapeutically active agent.

The combination referred to above may conveniently be presented for use in the form of a pharmaceutical formulation and thus pharmaceutical formulations comprising a combination as defined above together with a pharmaceutically acceptable carrier thereof represent a further embodiment of the invention.

The compounds of the present invention and pharmaceutically acceptable derivatives thereof may be prepared by the methodology described hereinafter, constituting a further embodiment of this invention.

In one embodiment the present invention provides a method for the preparation of compound(s) of formula (I) or (Ia) from an appropriate starting material, for example compound(s) of formula (II):

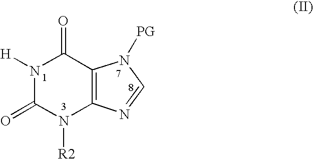

wherein PG=protecting group, the method comprising:
(i) alkylation at N1 of an N7 protected xanthine;
(ii) alkylation at N3 of an N7 protected xanthine;
(iii) halogenation at C8; and
(iv) de-protection of N7;
   in any order providing de-protection is carried out after alkylation.

### Process 1:

A process according to the invention for preparing compound(s) of formula (I) or (Ia) in which R1 incorporates a group selected from cycloalkenyl, heteroaryl and aryl.

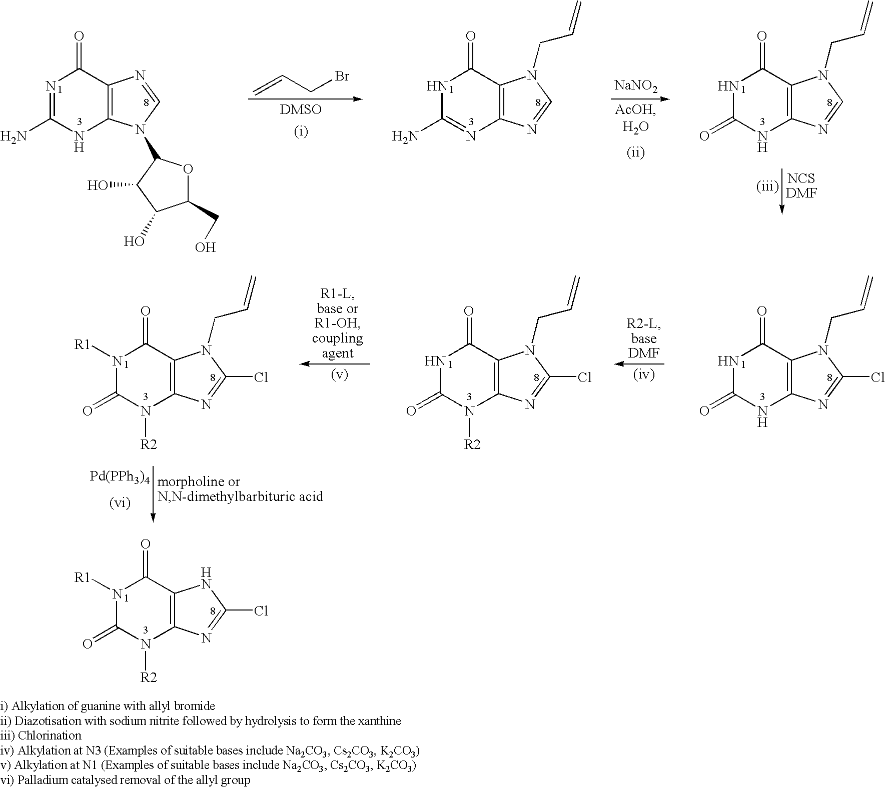

wherein L represents a leaving group, for example halogen.

### Process 2:

A process according to the invention for preparing compound(s) of formula (I) or (Ia) in which R1 incorporates a group selected pyrazoles, imidazole and tetrazole analogues and q is 1 or 2.

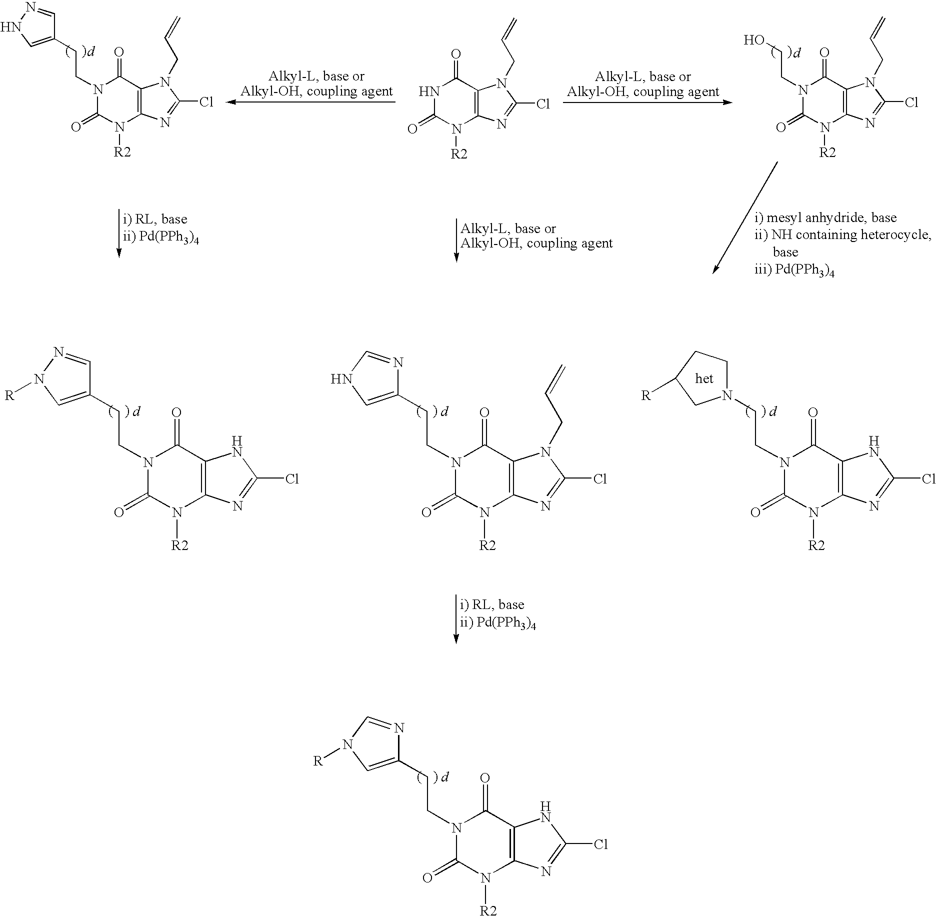

wherein L represents a leaving group, for example halogen, d represents (q-1), and R represents hydrogen or an optional substituent.

### Process 3:

A process according to the invention for preparing compound(s) of formula (I) or (Ia) in which R1 incorporates a group selected from oxadiazoles and q is 1 or 2.

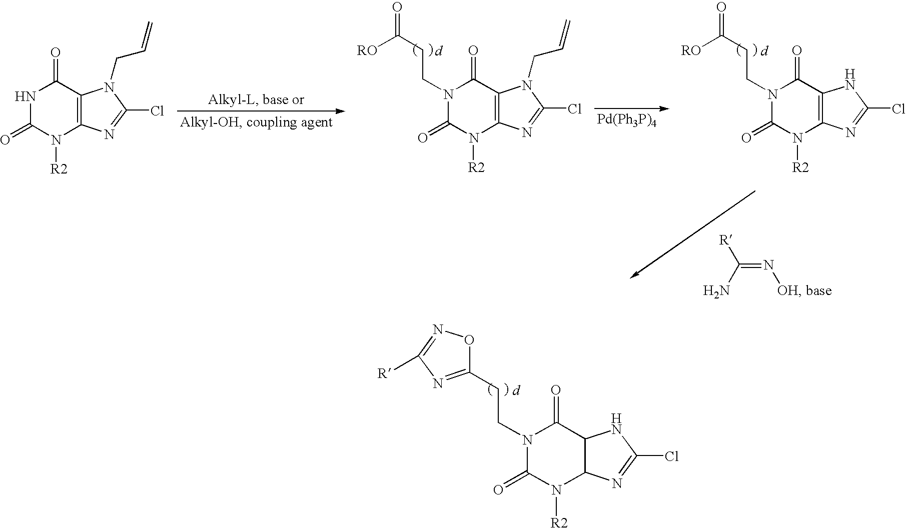

wherein L represents a leaving group, for example halogen, d represents (q-1), R represents an alkyl group and R′ represents hydrogen or an optional substituent.

### Process 4:

A process according to the invention for preparing compound(s) of formula (I) or (Ia) in which R1 incorporates a group selected oxadiazoles and q is 1 or 2.

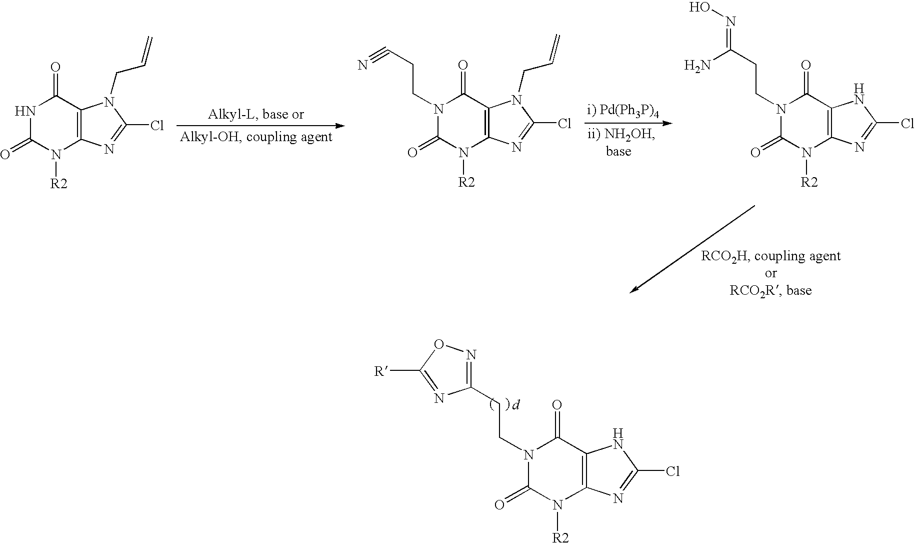

wherein L represents a leaving group, for example halogen, d represents (m-1), R represents an alkyl group and R′ represents hydrogen or an optional substituent.

### Process 5:

A process according to the invention for preparing compound(s) of formula (I) or (Ia) in which R³ is CN comprises steps (i) and (ii) of Process 1 followed by:

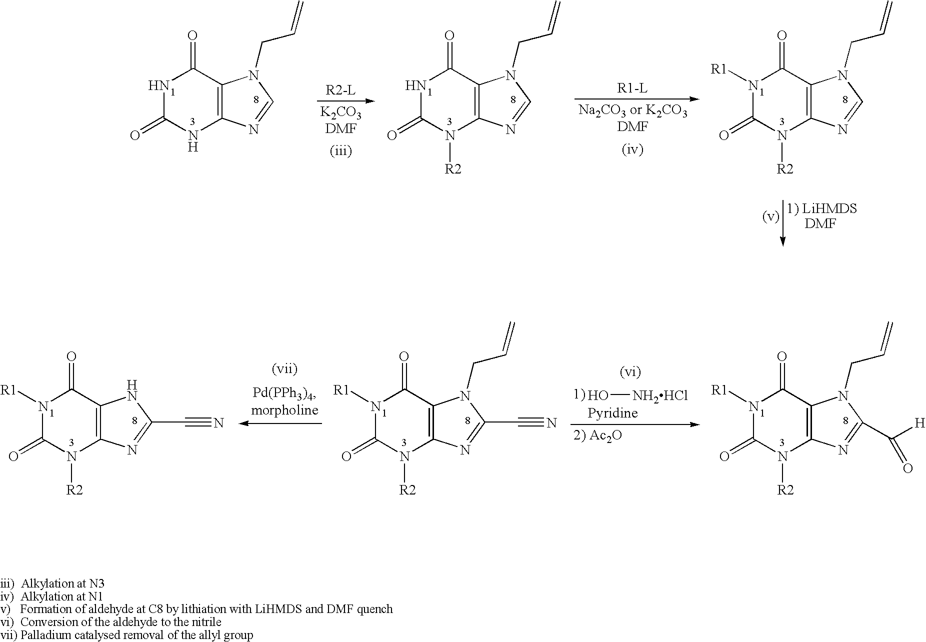

wherein L represents a leaving group.

### Process 6:

A process according to the invention for preparing compound(s) of formula (I) or (Ia) in which R³ is Cl or Br comprises steps (i) to (iv) of Process 5 followed by:

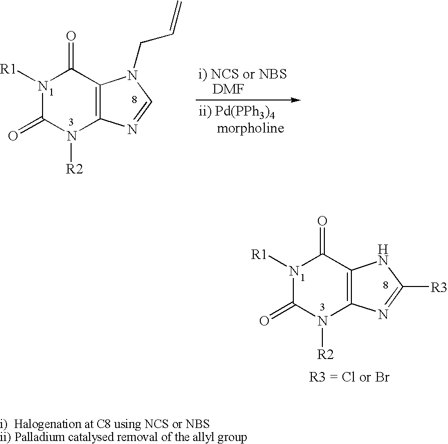

### Process 7:

A process according to the invention for preparing compound(s) of formula (I) or (Ia) in which R³ is CN comprises steps (i) to (iv) of Process 5 followed by:

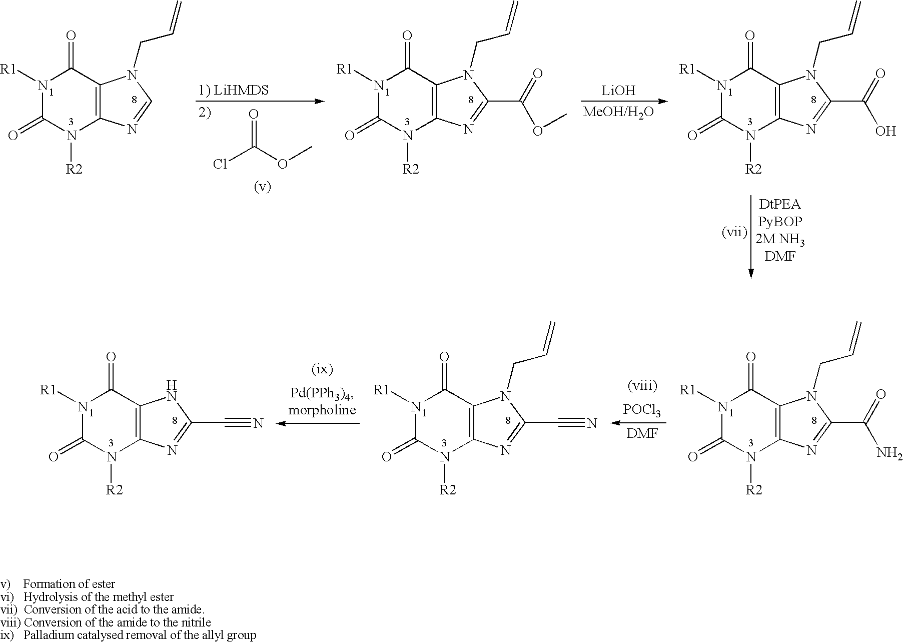

### Process 8:

A process according to the invention for preparing compound(s) of formula (I) or (Ia) in which R³ is Cl comprises:

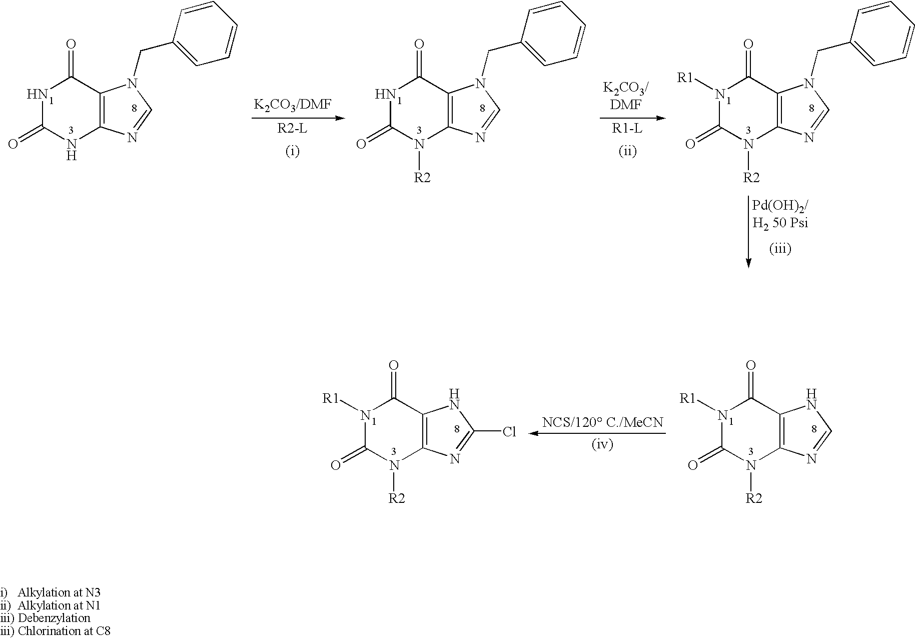

wherein L represents a leaving group

### Process 9:

A process according to the invention for preparing compound(s) of formula (I) or (Ia) in which R¹ differs from R², and R³ is Cl comprises steps (i) to (v) of Process 1 (where R² from Process 1 is specifically SEM or MEM) followed by:

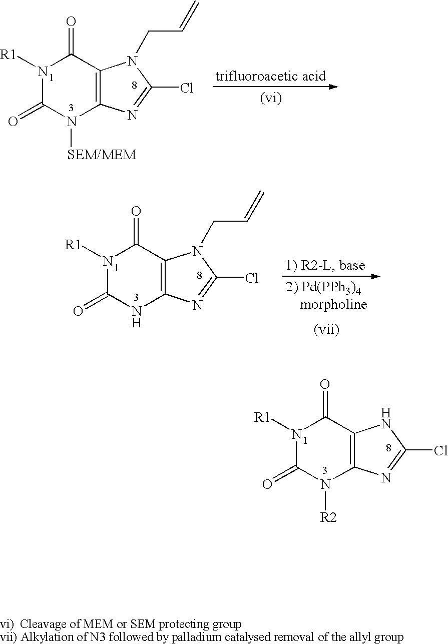

wherein L represents a leaving group

### Process 10:

A process according to the invention for preparing compound(s) of formula (I) or (Ia) in which R³ is Cl, Br or I comprises steps (i) to (iv) of Process 8 followed by:

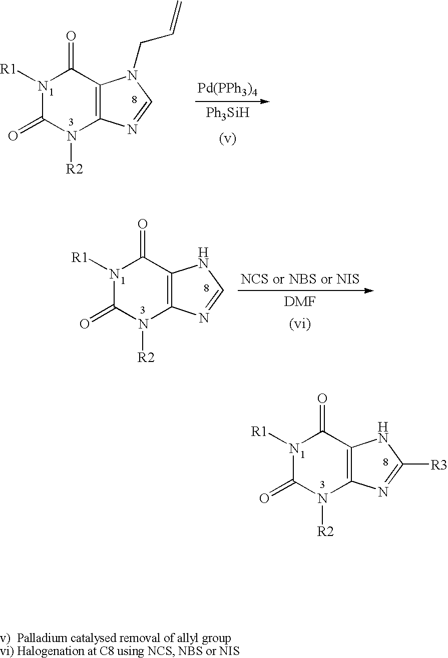

### Process 11:

A process according to the invention for preparing compound(s) of formula (I) or (Ia) comprises:

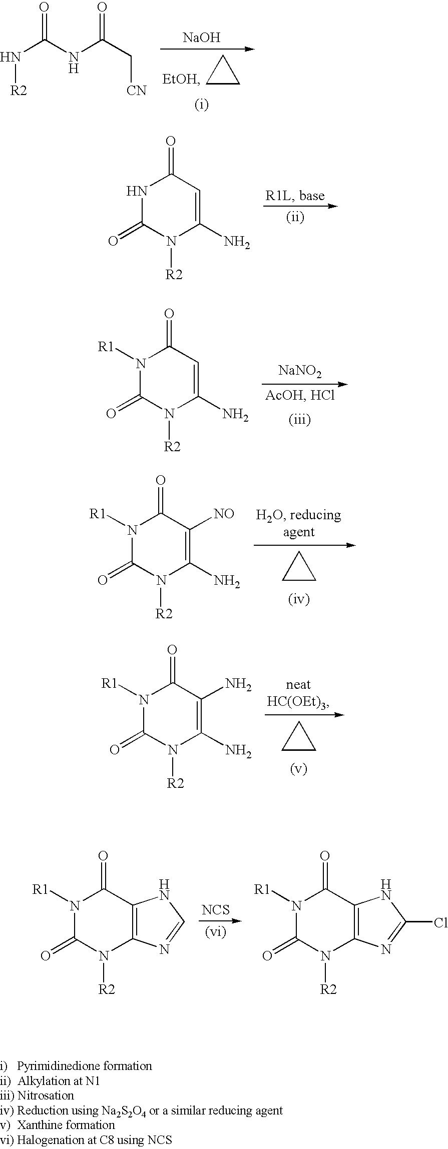

wherein L represents a leaving group

### Process 12:

A process according to the invention for preparing compound(s) of formula (I) or (Ia):

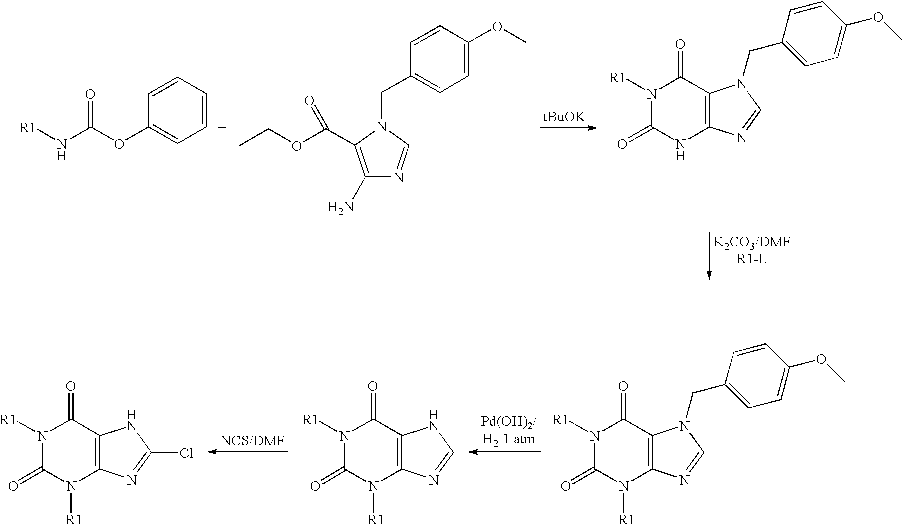

wherein L represents a leaving group.

Where desired or necessary, as a final stage in any of the above synthetic processes, a resultant compound of formula (I) or (Ia) can be converted into a pharmaceutically acceptable salt form or vice versa or converting one salt form into another pharmaceutically acceptable salt form.

### ABBREVIATIONS

AcOH Acetic acid

atm Atmosphere

br Broad (NMR)

CDI Carbonyldiimidazole

d Doublet (NMR)

DBAD Di-t-butylazodicarboxylate

DCM Dichloromethane

DIPEA Diisopropylethylamine

DMSO Dimethylsulfoxide

DMF N,N-Dimethylformamide

EtOAc Ethyl acetate

EtOH Ethanol

h Hour(s)

IPA Isopropyl alcohol

m Multiplet (NMR)

MDAP Mass directed autoprep

MeCN Acetonitrile

MeOH Methanol

min Minute(s)

NCS N-Chlorosuccinimide

NBS N-bromosuccinimide

NIS N-iodosuccinimide

q Quartet (NMR)

rt Room temperature

RT Retention time

Singlet (NMR)

SPE Solid phase extraction cartridge

t Triplet (NMR)

TFA Trifluoroacetic acid

THE Tetrahydrofuran

DMEM Dulbecco's Modified Eagle's Medium

HEPES 4-(2-Hydroxyethyl)piperazine-1-ethanesulphonic acid

LiHMDS Lithium hexamethyldisilylamide

Δ Heat

SEM 2-(trimethylsilyl)ethoxymethyl

MEM 2-methoxyethoxymethyl

The following non-limiting examples illustrate the present invention:

### SYNTHETIC EXAMPLES

### Example 1

### 8-Chloro-3-pentyl-1-phenyl-3,7-dihydro-1H-purine-2,6-dione

### a) 8-Chloro-3-pentyl-1-phenyl-3,7-dihydro-1H-purine-2,6-dione

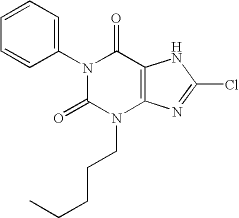

A solution of 3-pentyl-1-phenyl-3,7-dihydro-1H-purine-2,6-dione (51 mg, 0.17 mmol) in DMF (3 ml) was treated with NCS (25 mg, 0.19 mmol) and heated at 40° C. for 18 h. The solution was partitioned between 2M HCl (aq) and EtOAc. The organic layer was separated, washed with brine, dried (MgSO₄) and concentrated. The residue was taken up into MeOH and passed down an NH₂-propyl column (2 g). The product was eluted with 2% AcOH/MeOH and gave the title compound as a white solid (38 mg).

LCMS: m/z 333 [MH⁺], RT 3.2mins

¹H NMR (DMSO-d₆) δ: 0.86 (t, 3H, J=7 Hz), 1.30 (m, 4H), 1.67 (m, 2H), 3.92 (t, 2H, J=7 Hz), 7.26 (m, 2H), 7.45 (m, 3H), 14.58 (br s, 1H).

### b) 3-Pentyl-1-phenyl-3,7-dihydro-1H-purine-2,6-dione

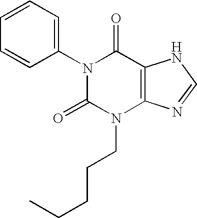

A solution of 7-{[4-(methyloxy)phenyl]methyl}-3-pentyl-1-phenyl-3,7-dihydro-1H-purine-2,6-dione (85 mg) in EtOH (10 ml) and AcOH (0.25 ml) was reacted with 10% Pd/C (50 mg) under an atmosphere of hydrogen for 18 h. The mixture was filtered through Celite® and the filtrate concentrated. Pd(OH)₂ (100 mg) was added and the reaction repeated. The title compound was obtained as a white solid (53 mg).

LCMS: m/z 299 [MH⁺], RT 2.7 mins.

### c) 7-{[4-(Methyloxy)phenyl]methyl}-3-pentyl-1-phenyl-3,7-dihydro-1H-purine-2,6-dione

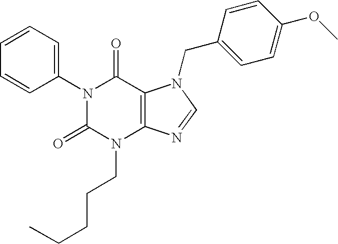

A solution of 7-([4-(methyloxy)phenyl]methyl)-1-phenyl-3,7-dihydro-1H-purine-2,6-dione (95 mg, 0.27 mmol), in DMF (4 ml) was treated with Na₂CO₃ (32 mg, 0.30 mmol) and 1-iodopentane (430, 0.33 mmol) then left to stir under N₂ for 3 days. The mixture was partitioned between 2M HCl (aq) and EtOAc. The organic layer was separated, washed with brine, dried (MgSO₄) and concentrated. The residue was purified by SPE (5 g) eluting with EtOAc/cyclohexane mixtures to give the title compound as an off-white solid (89 mg).

LCMS: m/z 419 [MH⁺], RT 3.5 mins.

### Example 2

### 8-Chloro-1-2-hydroxyphenyl)-3-pentyl-3,7-dihydro-1H-purine-2,6-dione

### a) 8-Chloro-1-(2-hydroxyphenyl)-3-pentyl-3,7-dihydro-1H-purine-2,6-dione

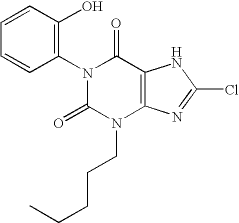

A solution of 1-(2-hydroxyphenyl)-3-pentyl-3,7-dihydro-1H-purine-2,6-dione (130 mg, 0.41 mmol) in DMF (4 ml) was treated with NCS (61 mg, 0.45 mmol) and stirred at rt. for 20 h. The solution was partitioned between 2M HCl (aq) and EtOAc. The organic layer was separated and washed with brine, dried (MgSO₄) and concentrated. Purification by MDAP gave the title compound as a white solid (45 mg).

LCMS: m/z 349 [MH⁺], RT 2.9 mins.

¹H NMR (DMSO-d₈) δ: 0.86 (t, 3H, J=7 Hz), 1.30 (m, 4H), 1.67 (m, 2H), 3.92 (t, 2H, J=7 Hz), 6.85 (m, 1H), 6.91 (m, 1H), 7.09 (m, 1H), 7.22 (m, 1H), 9.56 (s, 1H), 14.54 (br s, 1H).

### b) 1-(2-Hydroxyphenyl)-3-pentyl-3,7-dihydro-1H-purine-2,6-dione

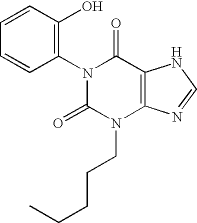

7-{[4-(Methyloxy)phenyl]methyl}-3-pentyl-1-{2-[(phenylmethyl)oxy]phenyl}-3,7-dihydro-1H-purine-2,6-dione (240 mg) was dissolved in EtOH (20 ml) and reacted with Pd(OH)₂ under an atmosphere of hydrogen. The mixture was filtered through Celite® and the filtrate concentrated, giving a grey solid (137 mg).

¹H NMR (DMSO-d₆) δ: 0.86 (t, 311, J=7 Hz), 1.30 (m, 4H), 1.69 (m, 2H), 3.98 (t, 2H, J=7 Hz), 6.85 (m, 1H), 6.91 (m, 1H), 7.08 (m, 1H), 7.22 (m, 1H), 8.06 (s, 1H), 9.55 (s, 1H), 13.63 (br s, 1H).

### c) 7-{[4-(Methyloxy)phenyl]methyl}-3-pentyl-1-{2-[(phenylmethyl)oxy]phenyl}-3,7-dihydro-1H-purine-2,6-dione

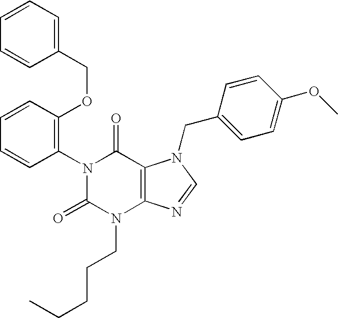

A solution of 7-{[4-(methyloxy)phenyl]methyl}-1-{2-[(phenylmethyl)oxy]phenyl}-3,7-dihydro-1H-purine-2,6-dione (250 mg, 0.55 mmol) in DMF (6 ml) was treated with 1-iodopentane (864, 0.66 mmol) and Na₂CO₃. The mixture was stirred at RT. under N₂ for 24 h. Further 1-iodopentane (294) was added and left for 18 h. The mixture was partitioned between 2M HCl (aq) and EtOAc. The organic layer was separated, washed with brine, dried (MgSO₄) and concentrated. The residue was purified by Companion eluting with EtOAc/cyclohexane mixtures to give the title compound as a white solid (240 mg).

LCMS: m/z 525 [MH₊], RT 3.6 mins.

### d) 7-{[4-(Methyloxy)phenyl]methyl}-1-{2-[(phenylmethyl)oxy]phenyl}-3,7-dihydro-1H-purine-2,6-dione

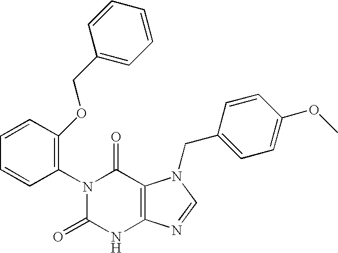

A solution of ethyl 4-amino-1-{[4-(methyloxy)phenyl]methyl}-1H-imidazole-5-carboxylate (1.0 g, 3.6 mmol) and phenyl {2-[(phenylmethyl)oxy]phenyl}carbamate (1.4 g, 4.4 mmol) in diglyme (15 ml) at 80° C., under N₂, was treated with the dropwise addition of a solution of KOt-Bu (1.6 g, 14.5 mmol) in diglyme (10 ml) over 30 mins. The mixture was left to react for a further 2 h then allowed to cool to rt. The mixture was partitioned between 2M HCl (aq) and EtOAc. The organic layer was separated, washed with brine, dried (MgSO₄) and concentrated. The residue was purified by Companion eluting with EtOAc/cyclohexane mixtures to give a brown solid. Et₂O was added and the title compound collected as a beige solid (653 mg).

LCMS: m/z 455 [MH⁺], RT 3.1 mins.

### e) Phenyl {2-[(phenylmethyl)oxy]phenyl}carbamate

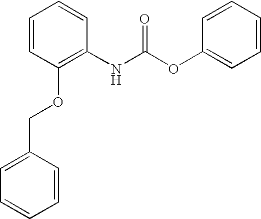

A solution of 2-benzyloxyaniline (2 g, 10.0 mmol) in THF (20 ml) was treated with the dropwise addition of phenylchloroformate (1.26 ml, 10.0 mmol) and stirred at rt for 10 mins. The mixture was partitioned between 2M HCl (aq) and EtOAc. The organic layer was separated and washed with brine, dried (MgSO₄) and concentrated, giving the title compound as a red/brown solid (2.89 g).

LCMS: m/z 320 [MH⁺], RT 3.6 mins.

### Example 3

### 3-Butyl-8-chloro-1-(phenylmethyl)-3,7-dihydro-1H-purine-2,6-dione

### a) 3-Butyl-8-chloro-1-(phenylmethyl)-3,7-dihydro-1H-purine-2,6-dione

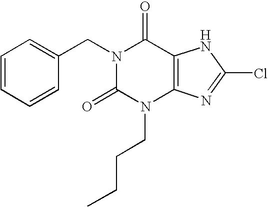

3-Butyl-1-(phenylmethyl)-3,7-dihydro-1H-purine-2,6-dione (100 mg, 0.336 mmol) and N-chlorosuccinimide (45 mg, 0.336 mmol) were suspended in MeCN (5 ml) and heated for 10 minutes at 120° C. under microwave irradiation. The reaction mixture was concentrated under reduced pressure and the title compound isolated using HPLC.

m/z 333 [Mh⁺].

¹H NMR (CDCl₃) δ: 0.96 (t, 3H, J=7 Hz), 1.41 (m, 2H), 1.75 (m, 2H), 4.10 (t, 2H, J=7.5 Hz), 5.26 (s, 2H), 7.30 (m, 3H), 7.52 (m, 2H), 13.00 (br s, 1H).

### b) 3-Butyl-1-(phenylmethyl)-3,7-dihydro-1H-purine-2,6-dione

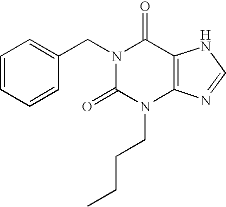

3-Butyl-1,7-bis(phenylmethyl)-3,7-dihydro-1H-purine-2,6-dione (0.556 g, 1.43 mmol) was dissolved in acetic acid (35 ml). Pd(OH)₂ on carbon (0.339 g) was added, and the mixture was shaken under hydrogen (at 50 psi) overnight. The catalyst was removed by filtration and washed with acetic acid. Fresh catalyst was added and the mixture was shaken under hydrogen (at 50 psi) overnight. The catalyst was removed by filtration and the filtrate was concentrated under reduced pressure to yield the title compound.

m/z 299 [MH⁺].

### c) 3-Butyl-1,7-bis(phenylmethyl)-3,7-dihydro-1H-purine-2,6-dione

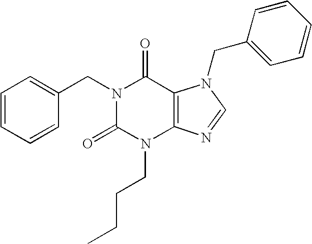

3-Butyl-7-(phenylmethyl)-3,7-dihydro-1H-purine-2,6-dione (0.50 g, 1.68 mmol) and potassium carbonate (0.347 g, 2.5 mmol) were suspended in DMF and benzyl bromide (0.233 ml, 1.96 mmol) was added. The reaction mixture was stirred at ambient temperature overnight. The reaction mixture was concentrated to dryness before suspending in ethyl acetate and washing with water followed by brine. The organic layer was dried over anhydrous sodium sulphate, filtered and concentrated under reduced pressure to yield the title compound.

m/z 389 [MH⁺].

### d) 3-Butyl-7-(phenylmethyl)-3,7-dihydro-1H-purine-2,6-dione

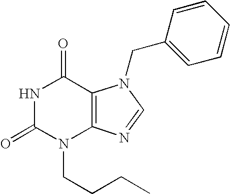

A suspension of 7-benzyl-3,7-dihydro-1H-purine-2,6-dione (5.0 g, 21 mmol) and potassium carbonate (3.34 g, 24 mmol) was stirred in DMF (125 ml) at 40° C. for thirty minutes before butyl iodide (2.55 ml, 23 mmol) was added. The mixture was stirred at 40° C. overnight. 50% Aqueous acetic acid (60 ml) was added, and the solution was concentrated under reduced pressure. The crude material was triturated with hexane before purifying using flash chromatography eluting with 1% methanol in dichloromethane to provide the product (3.55 g, 56%).

m/z 299 [MH⁺].

### Example 4

### 3-Butyl-8-chloro-1-(2-phenylethyl)-3,7-dihydro-1H-purine-2,6-dione

3-Butyl-8-chloro-1-(2-phenylethyl)-3,7-dihydro-1H-purine-2,6-dione was prepared similarly to 3-butyl-8-chloro-1-(phenylmethyl)-3,7-dihydro-1H-purine-2,6-dione using (2-bromoethyl)benzene.

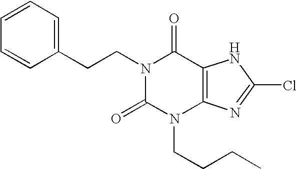

m/z 347.4 [MH⁺].

¹H NMR (CDCl₃) δ: 0.97 (t, 3H, J=7.5 Hz), 1.41 (m, 2H), 1.75 (m, 2H), 2.99 (t, 2H, J=8 Hz), 4.11 (t, 2H, J=7.5 Hz), 4.30 (t, 2H, J=8 Hz), 7.31, (m, 5H), 12.85 (br s, 1H).

All publications, including but not limited to patents and patent applications, cited in this specification are herein incorporated by reference as if each individual publication were specifically and individually indicated to be incorporated by reference herein as though fully set forth.

1. At least one chemical entity selected from compounds of formula (I) 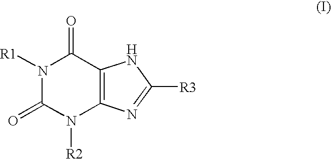
and pharmaceutically acceptable derivatives thereof, wherein
R¹ represents a group selected from
—(CH₂)_{q}-cycloalkenyl, —(CH₂)_{q}-aryl and —(CH₂)_{q}-heteroaryl;
Wherein if q is an integer selected from 1 or 2, the R¹ ring may be substituted by one or more groups independently selected from
C₁₋₆alkyl, C₂₋₆alkenyl, C₂₋₆alkynyl, halogen, —NH₂, —(CH₂)_{q}—(O)ₚ—(CH₂)_{q}—N(R⁵)C(O)OR⁸, —(CH₂)_{q}—N(R⁵)C(O)R⁸, —(CH₂)_{q}—(O)ₚ—(CH₂)_{q}—C(O)NR⁵R⁶, —(CH₂)_{q}—N(R⁵)C(O)N(R⁵)R⁶, —(CH₂)_{q}—C(O)N((CH₂)ₘOH)R⁵, —(CH₂)_{q}—N(R⁵)—S(O)₂R⁸, —CH₂—S(O)₂N(R⁵)R⁶, —OCF₃, —OCH(F)₂, —OCH₂F, —R⁸CN, CN and —SO₂R⁹;
Wherein if q is 0, the R¹ ring may be substituted by one or more groups independently selected from C₁₋₆alkyl, C₂₋₆alkenyl, C₂₋₆alkynyl, halogen, —NH₂, —(CH₂)_{q}(O)ₚ—(CH₂)_{q}—N(R⁵)C(O)OR⁸, —(CH₂)_{q}—N(R⁵)C(O)R⁸, —(CH₂)_{q}—(O)ₚ—(CH₂)_{q}—C(O)NR⁵R⁶, —(CH₂)_{q}—N(R⁵)C(O)N(R⁵)R⁶, —(CH₂)_{q}—C(O)N((CH₂)ₘOH)R⁵, —(CH₂)_{q}—N(R⁵)—S(O)₂R⁸, —CH₂—S(O)₂N(R⁵)R⁶, —C₁₋₆ haloalkyl, —OCF₃, —OCH(F)₂, —OCH₂F, —C(O)OR⁵, —OR⁵, —R⁸CN, CN, —SO₂R⁹; —(CH₂)ₙheteroaryl, —(CH₂)ₙheterocycyl, —(CH₂)ₙcycloalkyl, —(CH₂)ₙcycloalkenyl, and —(CH₂)ₙaryl;
R² represents a group selected from hydrogen, C₁₋₁₀alkyl, C₂₋₁₀alkenyl, C₂₋₁₀alkynyl, cycloalkyl, cycloalkenyl heterocyclyl, aryl, and heteroaryl, each of which may be optionally substituted by one or more of: C₁₋₁₀alkyl, C₂₋₁₀alkenyl, C₂₋₁₀alkynyl, cycloalkyl, heterocyclyl, aryl, heteroaryl, C₁₋₆ haloalkyl, halogen, —CN, —OR⁴, —(CH₂)ₙCOR⁴, —CO₂R⁴, —OCOR⁴, —(CH₂)ₙNR⁵R⁶, —(NH)ₚCONR⁵R⁶, —OCONR⁵R⁷, and —NHC(O)OR⁷;
R³ represents a group selected from halogen and CN;
R⁴ represents a group selected from hydrogen, C₁₋₆ alkyl, C₂₋₆alkenyl, C₂₋₆alkynyl, —(CH₂)ₙcycloalkyl, —(CH₂)ₙcycloalkenyl, —(CH₂)ₙheterocyclyl, —(CH₂)ₙaryl, and —(CH₂)ₙheteroaryl;
R⁵ and R⁶ are independently selected from hydrogen and C₁₋₄alkyl;
R⁷ represents a group selected from H, C₁₋₆alkyl, C₂₋₆ alkenyl, C₂₋₆alkynyl, —(CH₂)ₜcycloalkyl, —(CH₂)ₙcycloalkenyl, —(CH₂)ₜheterocyclyl, —(CH₂)ₜaryl, and —(CH₂)ₜheteroaryl;
R^{B} represents a group selected from C₁₋₄;
m represents an integer selected from 1, 2, 3, 4 and 5;
n represents an integer selected from 0, 1, 2, 3, 4 and 5;
t represents an integer selected from 1 and 2;
p represents an integer selected from 0 and 1;
q represents an integer selected from 0, 1 and 2.

2. At least one chemical entity according to claim 1 wherein R¹ is selected from —(CH₂)_{q}—aryl.

3. At least one chemical entity according to claim 1 or 2 wherein R² is selected from C₃₋₆ alkyl.

4. At least one chemical entity according to any preceding claim wherein R³ represents halogen.

5. At least one chemical entity according to any preceding claim wherein R³ represents chlorine.

6. At least one chemical entity according to any preceding claim for use in human or veterinary medicine.

7. At least one chemical entity according to any one of claims 1 to 5, for use in the treatment of disorders of lipid metabolism including dyslipidaemia and hyperlipoproteinaemia and/or of inflammatory diseases or conditions.

8. At least one chemical entity according to any one of claims 1 to 5 for use in the treatment of diabetic dyslipidaemia, mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa, obesity, coronary artery disease, thrombosis, angina, chronic renal failure, peripheral vascular disease or stroke.

9. At least one chemical entity according to any one of claims 1 to 5 for use in the manufacture of a medicament for treating diabetic dyslipidaemia, mixed dyslipidaemia, heart failure, hypercholesteraemia, cardiovascular disease including atherosclerosis, arteriosclerosis, and hypertriglyceridaemia, type II diabetes mellitus, type I diabetes, insulin resistance, hyperlipidaemia, anorexia nervosa, obesity, coronary artery disease, thrombosis, angina, chronic renal failure or stroke.

10. A method for the treatment of a human or animal subject having a condition where under-activation of the HM74A receptor contributes to the condition or where activation of the receptor will be beneficial, which method comprises administering to said human or animal subject an effective amount of at least one chemical entity according to any one of claims 1 to 5.

11. A method according to claim 10 wherein the human or animal subject has a disorder of lipid metabolism including dyslipidaemia or hyperlipoproteinaemia or an inflammatory disease or condition.

12. A pharmaceutical formulation comprising at least one chemical entity according to any one of claims 1 to 5 and at least one pharmaceutically acceptable diluents, excipients or carriers.

13. A combination for administration together or separately, sequentially or simultaneously in separate or combined pharmaceutical formulations, said combination comprising at least one chemical entity according to any one of claims 1 to 5 together with another therapeutically active agent.

14. A pharmaceutical formulation comprising:
(i) at least one chemical entity according to any one of claims 1 to 5;
(ii) one or more active ingredients selected from statins, fibrates, bile-acid binding resins and nicotinic acid; and
(iii) one or more pharmaceutically acceptable diluents, excipients or carriers.
